(19) United States
(12) Patent Application Publication
(10) Pub. No.: US 2018/0172663 A1
(43) Pub. Date: Jun. 21, 2018

(54) A MEMS THROMBELASTOGRAPH/VISCOELASTICITY ANALYZER

(71) Applicant: ATANTARES CORP., Cambridge, MA (US)

(72) Inventor: Qi Qin, Arlington, MA (US)

(73) Assignee: ATANTARES CORP., Arlington, MA (US)

(21) Appl. No.: 15/737,258

(22) PCT Filed: Jun. 14, 2016
(86) PCT No.: PCT/US16/37433

§ 371 Date: Dec. 15, 2017

Related U.S. Application Data
(60) Provisional application No. 62/175,610, filed on Jun. 15, 2015.

(51) Int. Cl. G01N 33/49; G01N 33/487; G01N 11/14; B81B 7/02

(52) U.S. Cl. CPC G01N 33/49; B81B 7/02; G01N 11/14; G01N 33/48785

(57) ABSTRACT

Disclosed herein is a micro thrombelastograph micro TEG which can be used in clinic, research and POC (point of care) settings. The present disclosure relates to a miniaturization method of traditional thrombelastograph assay based on the micro electro-mechanical system (MEMS) technology platform. In addition, this disclosure can be applied to general viscosity/rheology measurement.

### CROSS-REFERENCE TO RELATED APPLICATION

This patent application claims priority to and the benefit of United States provisional patent application serial number U.S. Ser. No. 62/175,610, entitled “MEMS Thrombelastograph/Viscoelasticity Analyzer”, filed on Jun. 15, 2015, the entire contents of which are herein incorporated by reference.

### BACKGROUND

Thromboelastometry (TEM), previously named rotational thromboelastography (ROTEG) or rotational thromboelastometry (ROTEM), is an established viscoelastic method for hemostasis testing in whole blood. It is modification of traditional thromboelastography (TEG). TEM investigates the interaction of coagulation factors, their inhibitors, anticoagulant drugs, blood cells, specifically platelets, during clotting and subsequent fibrinolysis. The rheological conditions mimic the sluggish flow of blood in veins. TEM is performed with the ROTEM whole blood analyzer (Tem Innovations GmbH, Munich) and is an enhancement of thrombelastography, originally described by Hartert in 1948.

### SUMMARY

The subject invention comprises a method and apparatus to make miniaturized/portable and robust instruments for thromboelastometry. The invention uses MEMS technology to integrate rotation driver, position/force sensor and sensing pole in a single embodiment. This device is made by silicon fabrication technology and can be as small as several millimeters. The device is operated totally electrically. Thus it can be very compact and good for POC application. In addition, its natural frequency can be far away from its operation frequency (1-10 Hz), resulting in a robust system. Furthermore, the invention can be integrated with microfluidics system to achieve high throughput measurement which is highly desired by many user cases.

### BRIEF DESCRIPTION OF THE DRAWING

These and other features and advantages of the present invention will become more readily appreciated as the same becomes better understood by reference to the following detailed description when considered in connection with the accompanying drawings, wherein:

FIG. 1 is a fragmentary perspective view showing an exemplary uTEG device embodiment without a liquid chamber. This device includes spring suspension, capacitive actuation and sensing in top layers; middle layer (frame) for housing and protection; bottom layer(s) for spinning rod in contact with sample.

FIG. 2 is a simplified view showing an uTEG device mounted on a liquid chamber with inlet. This can be implemented in a microfluidics platform.

FIG. 3 shows some other possible spring suspension configurations.

FIG. 4 is the front evaluation view of four exemplary shaft configurations for viscosity/viscoelacity measurement.

FIG. 5 is also a fragmentary perspective view showing another exemplary uTEG device embodiment without a liquid chamber.

FIG. 6 is the top view as in FIG. 5.

FIG. 7 is the bottom view as in FIG. 5, but showing the bottom hollow shaft.

FIG. 8 is a simplified view showing another measurement configuration in which the liquid chamber rotates.

FIG. 9 is a flow chart for measurement sequence.

FIG. 10 is the process of u-TEG.

FIG. 11 is the schematics of assembled u-TEG.

### DETAILED DESCRIPTION OF CERTAIN EMBODIMENTS

The prior art are varieties version of enhanced thrombelastography. There are 3 instruments currently used in veterinary and human medicine; the Sonoclot coagulation and platelet function analyzer or Sonoclot (Sienco Inc., Arvada, Colo., USA), the TEG thrombelastograph hemostasis analyzer or TEG (Haemonetics Corporation, Braintree, Mass., USA), and the ROTEM (Pentapharm GmbH, Munich, Germany). The Sonoclot and ROTEM measure changes in impedance to movement of a vibrating probe immersed in a blood sample, whereas TEG utilizes an oscillating cup with a fixed probe or piston. The probe is a torsion wire in TEG technology, whereas an optical detector is used by the ROTEM. All 3 instruments measure the rate of fibrin formation, clot strength, and clot lysis. The TEG and ROTEM have become increasingly used in POC management of trauma and perioperative bleeding during cardiac and liver transplantation in people. In 1966 the Haemoscope Corporation (now part of Haemonectics Corporation) registered trademarks for the terms thrombelastography and TEG; thus, these terms are limited to evaluations done with the Haemoscope analyzers. Pentapharm GmbH has also registered trademarks for the terms ROTEM and thromboelastometry. The technology and data obtained are similar; for the purposes of this review “TE” will refer to analysis done with either the ROTEM or the TEG. ROTEM and TEG are prone to the external vibration. For example, according to ROTEM's user's report, the system has to be recalibrated after it is moved. So the system is very inconvenient for users on mobile.

Some new instruments in pipeline are targeting thromboelastometry. Haemonetics is testing and marketing its new product TEG® 6S which uses resonance phenomenon to measure thromboelastometry. TEG® 6S is for POC application and has sensors integrated in a cartridge. The new instrument is more compact and robust than the previous TEG. Some other devices use micro-cantilever to sense the change of blood viscoelasticity. Although these instruments can prove its correlation to the previous TEG, these methods have to prove their sensitivity before being accepted by market.

Furthermore, similar instruments with current TEG and ROTEM are used to measure viscosity and rheology. These instruments mostly use torsion wire to measure small forces. These prior arts usually are composed with components in macro scale. They are also prone to external vibration and expensive. Additionally, these instruments are bulky and it is difficult to make high throughput devices.

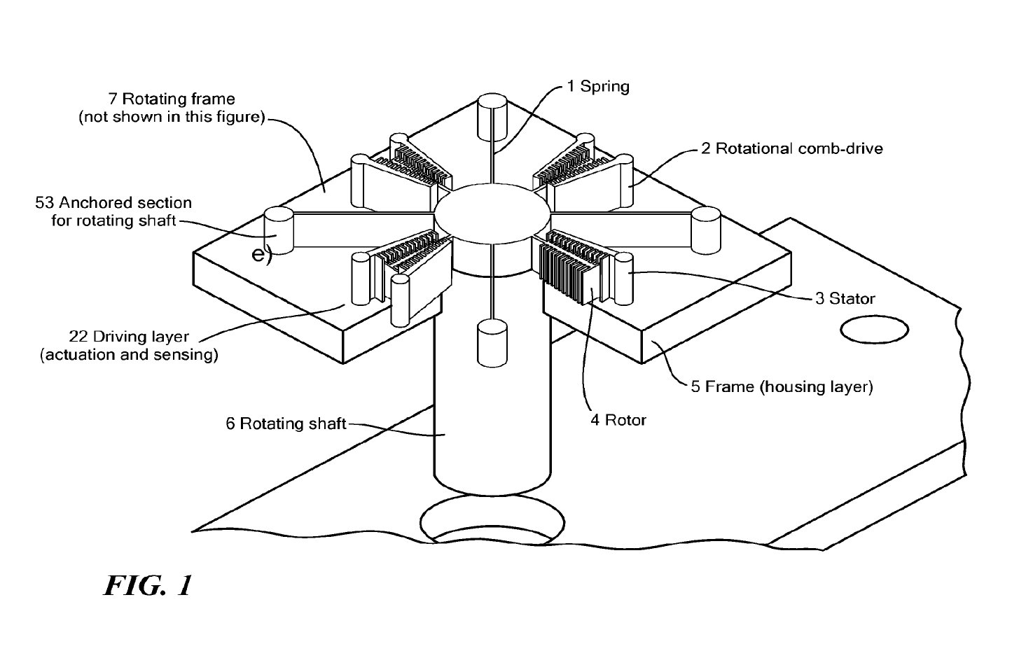

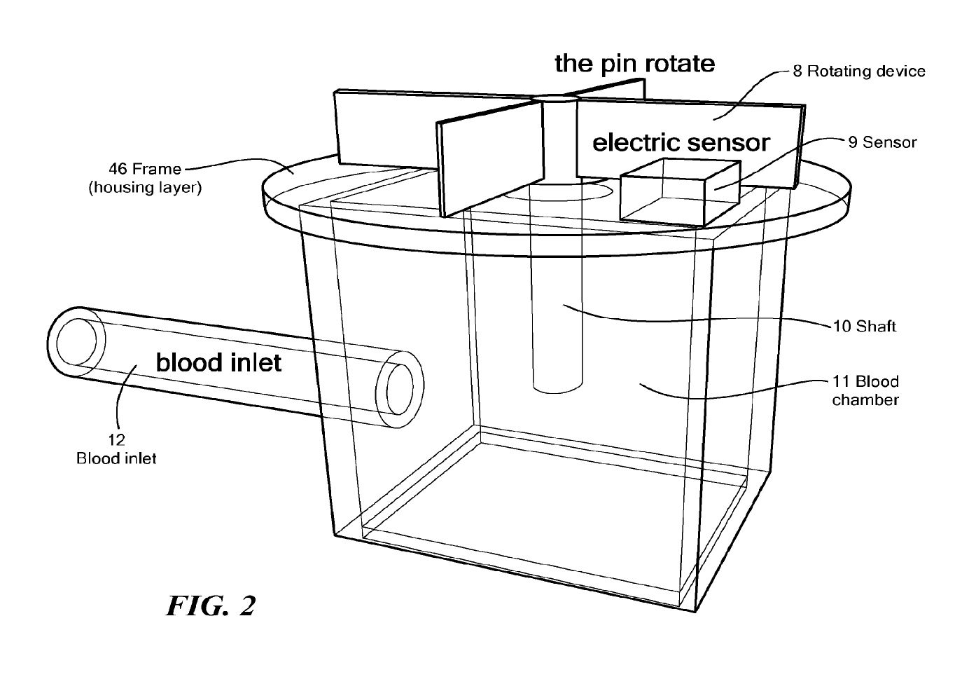

Referring to the FIGs., wherein like numerals indicate like or corresponding parts throughout the several views, an exemplary implementation of micro-TEG is generally shown in FIGS. 1 and 2. For purposes of illustration and not to be in any way limiting, the following description will make reference to the TEG application. However, it will be appreciated that the invention is equally applicable to other viscosity and rheology measurement using rotational configuration.

General speaking, there are two basic viscoelasticity measuring methods using rotational configuration. The first one provides a controlled stress input and determines the resulting shear rate. The dynamic response of this pin immersed in sample is sensed by a transducer and viscoelasticity is extracted. The second one generate a controlled shear rate input and determines the resulting shear stress. In the first method, a shaft (Bob), suspended by a torsional wire, is inserted into a sample and the cup is rotating. The force will be sensed by the rotating angle of torsional wire.

Typically, but not necessarily, as illustrated in FIG. 2, the device is composed with a blood container 11, a rotating driver 8, a rotating shaft 10 and a position/stress sensor 9. This device can be an independent unit or a part of a microfluidic systems for either portable or high throughput purpose. When it is integrated with micro-fluidics device, an inlet 12 is needed to inject blood or other liquid into the container. The rotating shaft 10 is driven by the rotating driver 8. The rotation of the shaft can be either oscillation or continuous rotation. The lower part of this shaft is immersed in blood or other liquids, the shear movement at the interface between the shaft and liquid generate force due to the viscosity or viscoelasticity of the measured liquids. This force changes the dynamics of the shaft which is recorded by the sensor 9. By analyzing the recorded data, the viscosity or viscoelastic of the measured liquid can be extracted precisely.

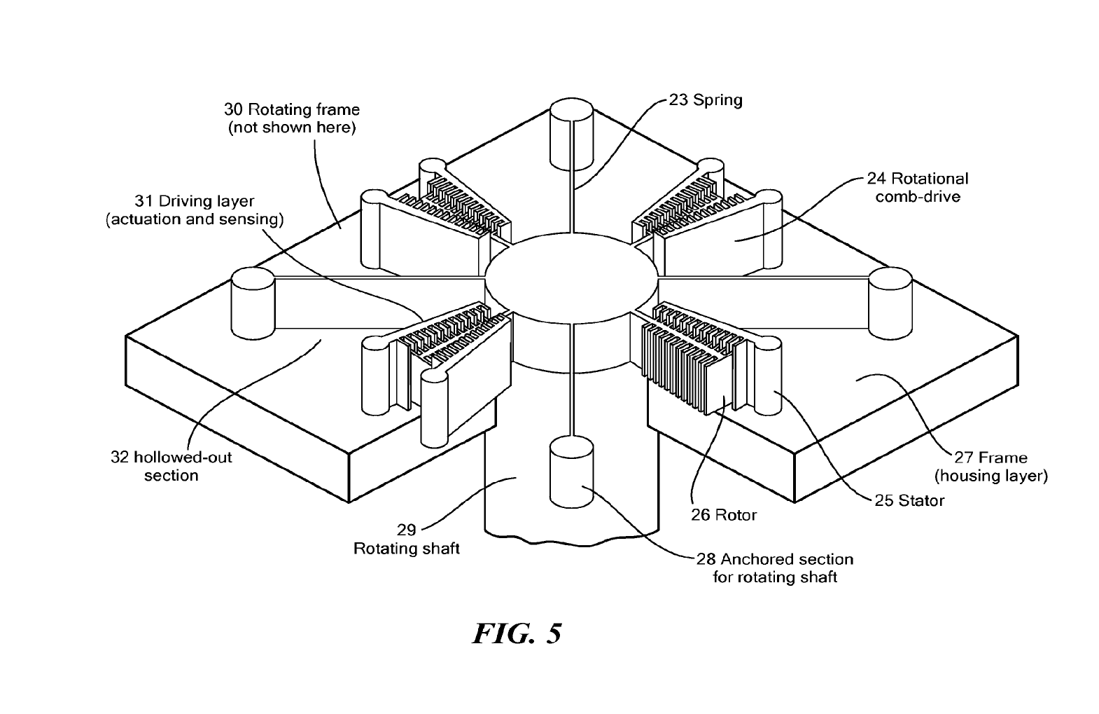

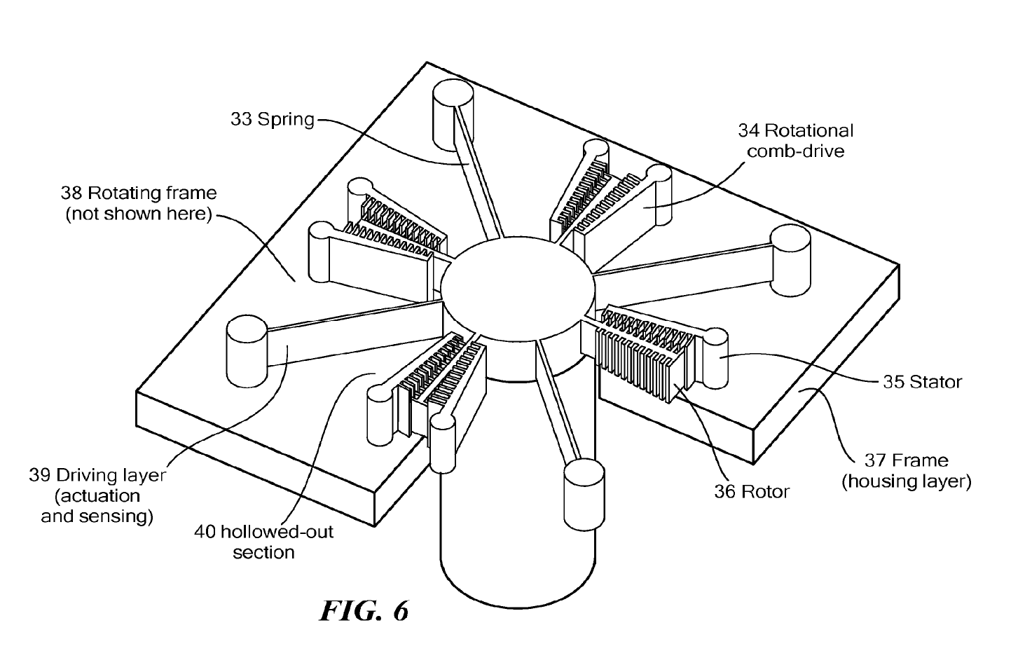

The rotation driver is made by MEMS. It can be either rotational comb-drives or other MEMS motors. Example embodiments with comb-drive are illustrated in FIGS. 1, 5, 7 and 8. The suspended rotors 4, 26, 36 of the comb-drive 2, 24, 34 are attached with the end of rotating shaft 6, 29, 45 at the anchor section 53, 28. The distribution of the comb-drives is radical symmetric (FIGS. 1, 5, 7 and 8). The stators are fixed. When applied voltage, the electrostatic force generated between the rotor and stator gives torque to the rotational shaft 6, 29, 45. Fingers of rotors and stators are curved to comply rotational movement. In addition, there are two sets of fingers for each comb of rotor. So during any actuation cycle, only one set of comb is biased with large voltage. The other set is used to sense the position of the structure. Another embodiment, which is not shown, have two sets of comb. One set is used to actuate rotation. The other is used to sense only. The size of the whole driver can be varied from several millimeter to centimeter depending on application requirement. A larger device can have more comb-drive fingers, leading to a greater driving force and more rotational displacement.

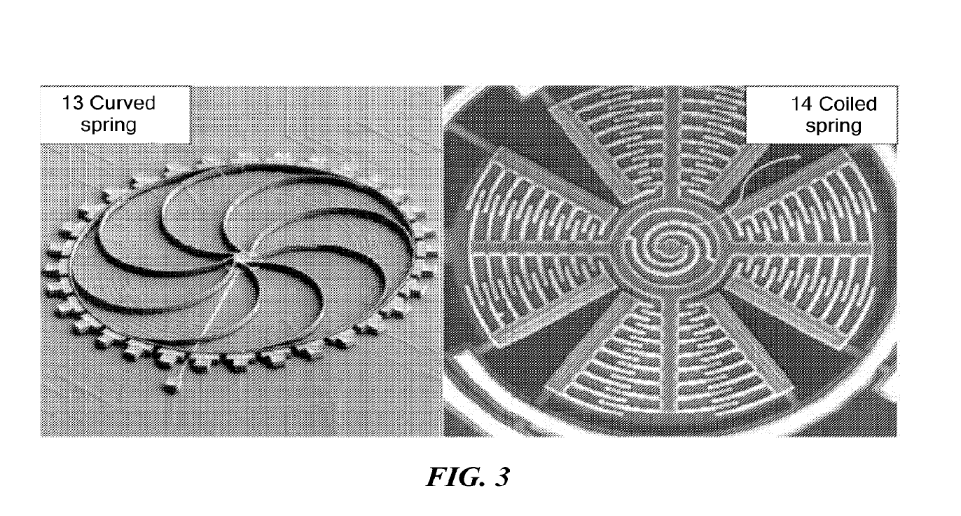

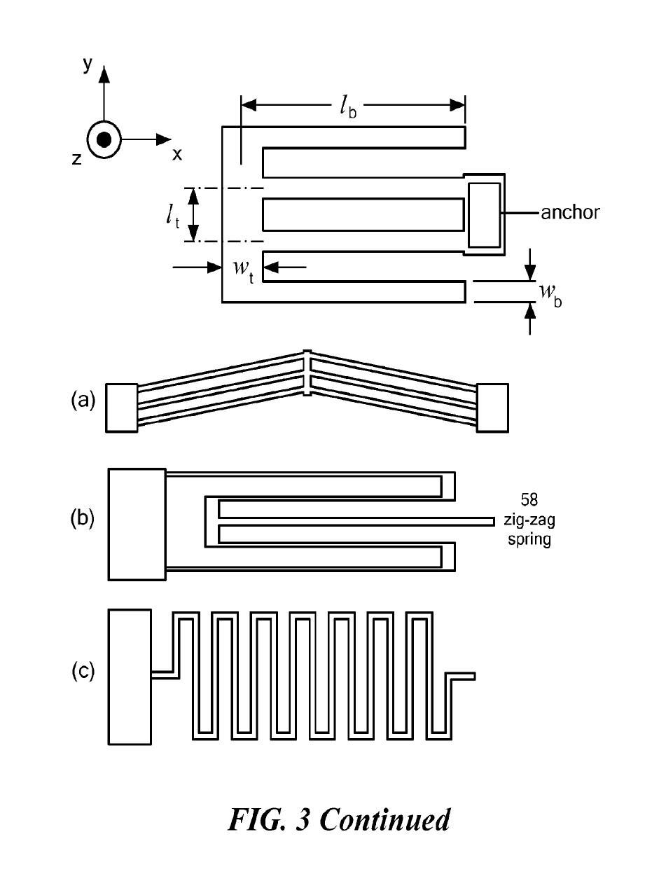

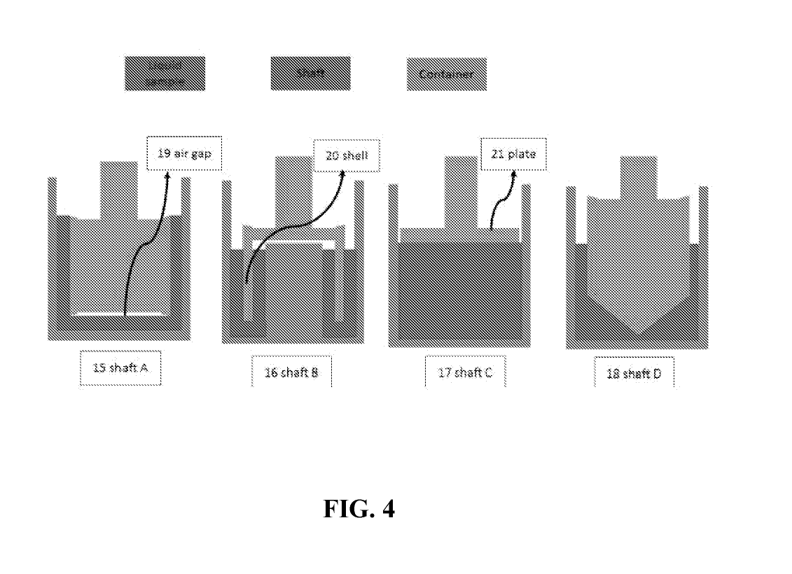

(spring and alternatives) Special designed spring 1 system are applied to confine the system movement in a rotational way. In this system, a number of springs are placed in a rotational symmetric configuration. One end of the spring is anchored and the other end is connected to the rotational shaft or rotating frame 7. So the rotational part is suspended and only supported by the spring. The spring system is designed in the way that the system has a large stiffness in the radius direction and is easy to rotate. So the system is sensitive to small torque and resistive to the perturbation in the radius direction. One of example embodiments is illustrated in FIG. 1. Here only a single beam is used to demonstrate the idea. In the real design, to soften springs, multi-fold spring can be used. The other possible implementations are shown in FIG. 3.

(design of the frame) There is a frame 5 below the spring and the comb-driver. This frame is fixed on the liquid container 11 and functions as a mechanical support. In is center is a hole 43 which let the rotational shaft to go through. In addition, the springs are anchored on this frame.

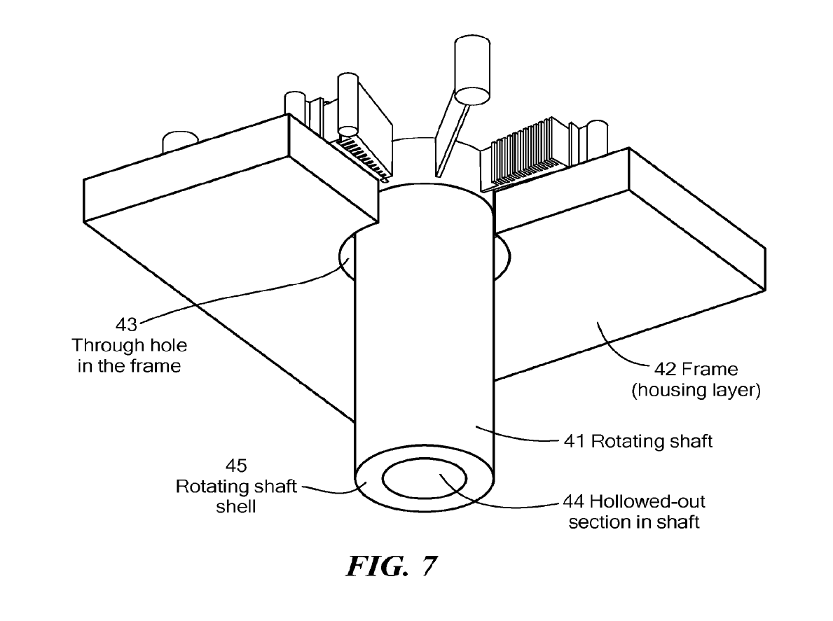

(shaft shape) Different shaft shapes are used to achieve pure shear rate measurement. In each of these designs, a thinner part is connected to the driving system above the frame 5. The larger part will eventually immersed in liquid samples. In design A, the top and bottom surfaces are recessed and the end effects from these two faces are eliminated. The air gap 19 between the recession and liquid can isolate sample and the shaft. In the design B, the lower part of the shaft has a shell 20 structure which eliminate the possible non-shear movement of the bottom surface when the shell rotates. In the design C, only a plate 21 is attached to the shaft bottom. The design D is inherited from Mooney-Ewart System. It allows the calculation of the bottom end surface by creating a flow in a double cone gap. The example implementation in FIG. 7 uses a shaft 45 similar with shaft B 16.

(package cap) On top of the driving layer 22, a cap layer is used to encapsulate the whole device. This cap layer can be either a glass wafer or silicon wafer. Electrical connections go through this cap layer to bias the comb-drive and sensors.

(sensor reading) The sensing of the shaft dynamics is achieved by the comb itself or by additional capacitor sensors. The driving system will rotate the shaft in a low frequency (1-20 Hz) for several degrees (1-5 degrees). So this movement can be considered quasi-static when a small high-frequency chopping signal is applied. This small signal is used to measure the total capacitance at a transient moment. The position of the comb and the rotation degree of the shaft can be extracted by this capacitor value. Thus the dynamics of the shaft can be analyzed. The other way is that additional sensing comb can be added to the system. This comb will not be biased as the driving comb-drive. It only acts as a capacitor sensor to sense the location and rotation.

An important objective of the system design is to achieve vibration proof. This can be translated to a high natural frequency of the mechanical system. This can be realized by reduce the mass of the movable components and increase spring constant. The spring constant, however, are constrained by the requirement that this constant should be small enough to sense the small viscosity/viscoelasticity force (the small viscosity/viscoelasticity force can generate enough rotational displacement). Mass reduction can be achieved by hollowing the layer 22, 31, 39 of comb-drive and the rotating shaft. Example embodiments are hollowed section 32, 40 and hollowed shaft 44.

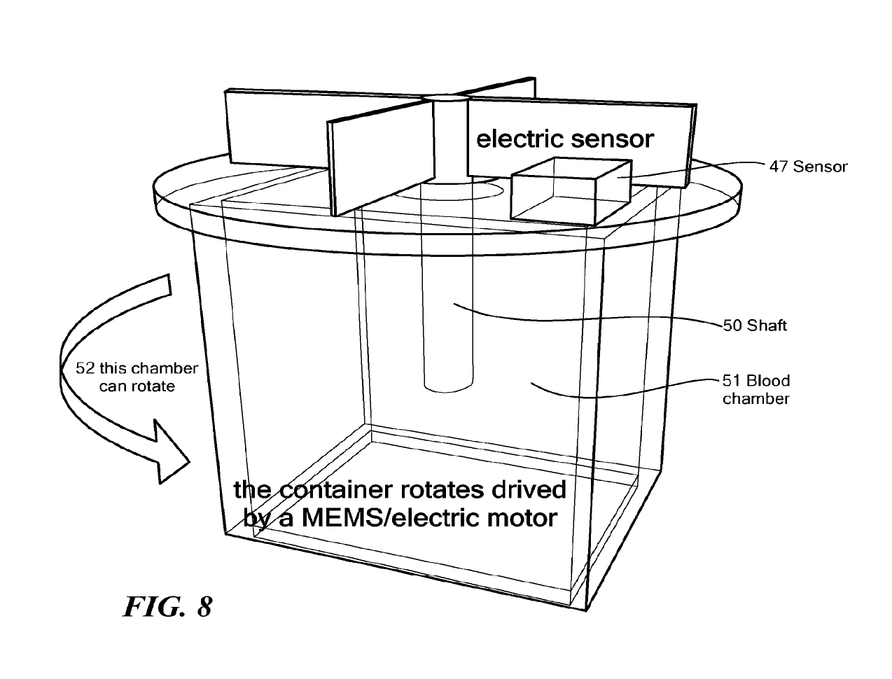

Another instrument implementation is illustrated in FIG. 8. In this method, instead of a driven rotating shaft, the sample container 51 rotates powered by either MEMS or other compact motors. A similar comb drive system shown in FIG. 1 is placed on top of the container. The shaft 50 is immersed in the sample and is driven by the viscosity force from the rotating sample. The movement is sensed by comb structures or other electric sensors. The recorded signal can be used to extract the information about the viscosity or viscoelasticity properties.

### EXEMPLIFICATION

The following examples illustrate some embodiments and aspects of the invention. It will be apparent to those skilled in the relevant art that various modifications, additions, substitutions, and the like can be performed without altering the spirit or scope of the invention, and such modifications and variations are encompassed within the scope of the invention as defined in the claims which follow. The following examples do not in any way limit the invention.

### Example 1

The present Example describes fabrication and characterization of a MEMS micro-TEG device as disclosed herein.

### Fabrication Process:

The comb drive part of the device is a 4 layer structure. Layer 1 is for the support to the cob drive fingers and electrodes. To minimize parasitic capacitance, glass will be the first choice for layer 1. Silicon can also be option if coated with an insulating layer. Layer 2 is for the comb drive. It will be made of silicon using DRIE. Layer 3 has shaft connecting the comb drive and the rotor in blood. It also provides support to the comb drive. Layer 4 has the rotor that is driven by the comb drive and spin in the blood to be tested.

The drive will be packaged to a blood container (not shown here). The blood container, alone or together with the comb drive, can be disposable, depending on the application need and cost consideration.

This process assumes batch fabrication of all layers of the comb drive for the sake of precision and low cost. In case precision can be relaxed and further cost reduction is required, only the comb layer will be made of Si out of MEMS batch fabrication, other layers can be replaced with cheaper materials such as plastics.

### Process for Layer 1:

Layer 1 only has some through holes in it. After through hole etching it should be bonded to layer 2.

The options for through hole etching (assuming glass material)
1. Deposit on both side a layer of poly silicon, pattern the wafer with photolithography, and etch through by wet process.
2. Ultrasound drilling
3. Laser drilling

If using silicon, layer 1 process will be photo lithography and deep reactive etching (DRIE), followed by coating the whole wafer with an insulating layer, such as silicon oxide, for electric isolation to the comb layer.

### Layer 2 Process

1. Start with a double side polished blank silicon wafer, and grow a layer of thermal oxide.
2. On both sides of the wafer, pattern with photo lithography process, etch oxide by reactive ion etch (RIE) or buffered oxide etcher (BOE), etch shallow clearance into Si by DRIE.
3. Deposit a thicker layer of oxide on top side.
4. Pattern top side with photo lithography process, etch through the oxide by BOE, and clean the wafer.
5. Strip bottom oxide.
6. Bond to layer 3 and layer 4 by fusion bonding.
7. Etch comb fingers by DRIE using the thicker oxide patter as mask, and etch through layer 3.
8. Strip top oxide.
9. Bond to layer 1 by anodic bonding

### Layer 3 Process

1. Start with a double side polished blank silicon wafer, and grow a layer of thermal oxide.
2. Pattern both side with photo lithography process, etch a shallow clearance on top by DRIE, etch the bottom using black silicon DRIE recipe, and clean the wafer.
3. Pattern both sides with photo lithography process, etch from top deep into the wafer by DRIE, leaving only a few tens of microns, etch through from bottom, and clean the wafer. By the end of the etches, there should be a few tabs holding the center shaft and aligned to the openings in layer 2 so these tab can be cleared to free the shaft after bonding to layer 2 by DRIE from top side of layer 2.
4. Bond to layer 2 and layer 4 by fusion bonding.
5. Etch through the tabs by DRIE from layer 2 top side to free the shaft.

### Layer 4 Process

1. Start with a double side polished blank silicon wafer, and grow a layer of thermal oxide.
2. Pattern with photo lithography process on both sides, etch top sides by regular DRIE recipe and follow with a black silicon recipe, DRIE etch the bottom side, and clean the wafer.
3. Bond to layer 2 and layer 4 by fusion bonding.
4. After etch through layer 2 and layer 3, strip silicon oxide on top of layer 2, and bond L1 to L2-4 stack.
5. Etch through from bottom side by DRIE.

### Bonding Process

1. Bond layer 2, layer 3 and layer 4 by fusion boding.
2. Etch through L2 using its oxide layer as mask.
3. Strip layer 2 oxide, make sure the layer 4 oxide has enough remaining for layer 4 through etch later.
4. Bond layer 1 to layer 2 top side by anodic bonding.
5. Etch through layer 4
6. Dicing
7. Attach individual comb drive dies to blood container

(Circuit reading) The control and readout circuit can be either integrated on the MEMS system or a separate component. The control unit is in charge of adding desired voltage to the comb-drive and the readout unit can add small chopped signal to sense the capacitance which can be used to calculate the moving dynamics.

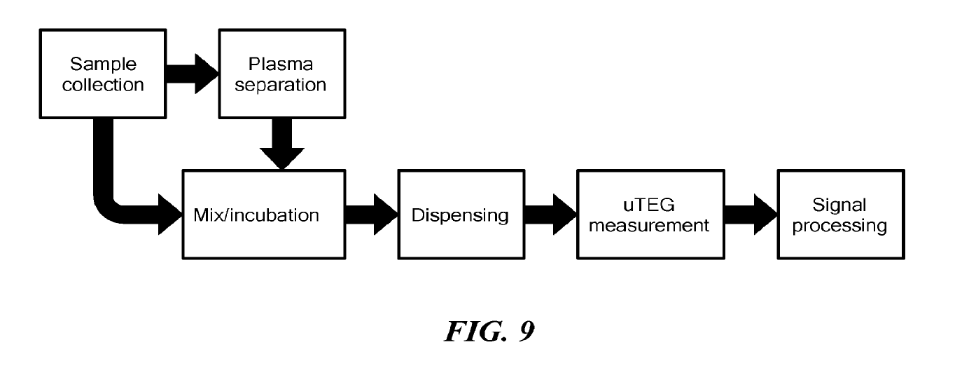

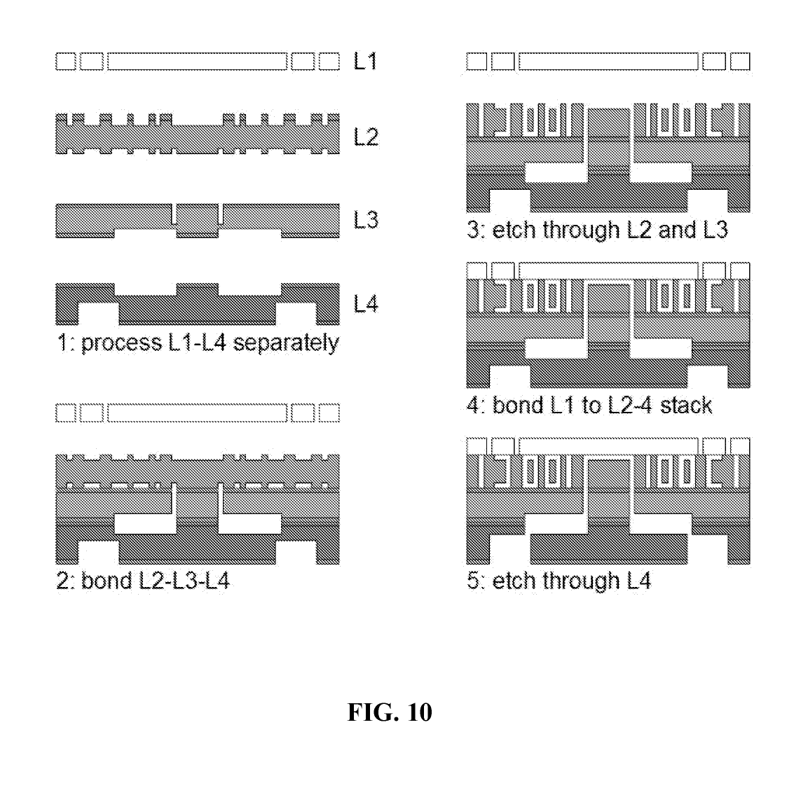

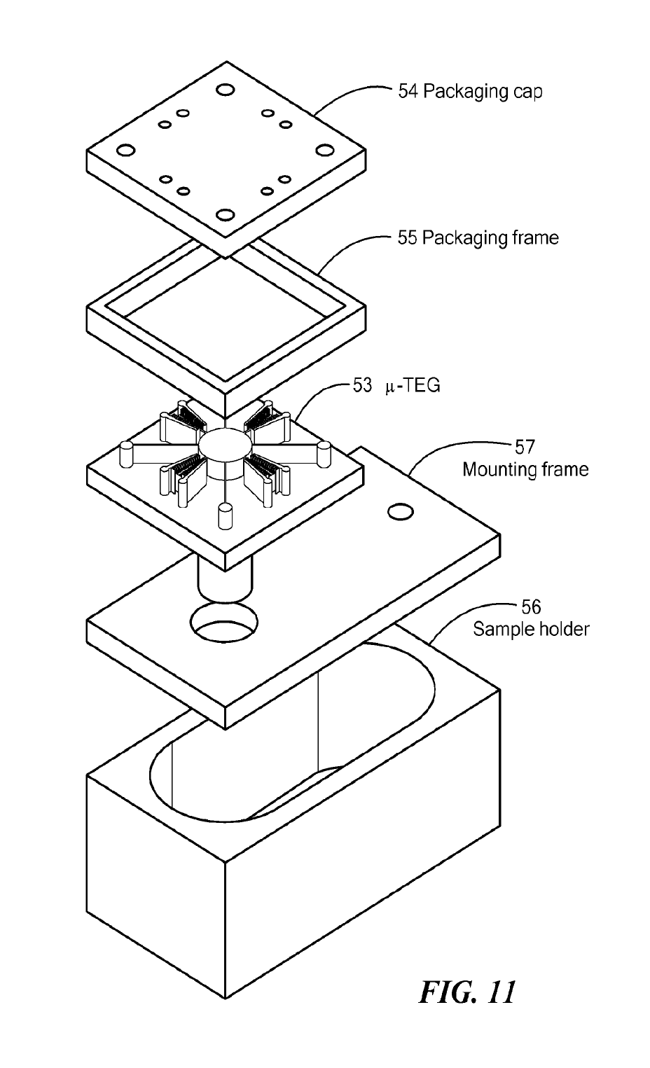

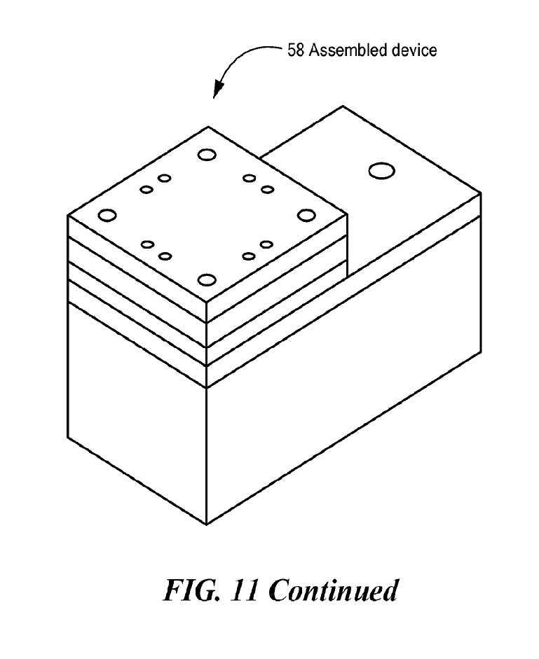

To measure Thromboelastometry or other liquid viscosity/viscoelasticity, the measuring sequence is illustrated in FIG. 9. Samples are collected and dispensed to the measurement system. If plasma is required, a plasma separation unit is added. If not, the collected samples are mixed/incubated with the requested reagents according to assay standard procedure. Then the mixture is dispensed into the measuring chamber illustrated in FIGS. 2 and 8. Thromboelastometry or liquid viscosity/viscoelasticity is measured.

The signal is processed by a computer and the final results are displayed.

What is claimed is:

1. A rotational viscosity/viscoelasticity measurement instrument design and its fabrication process as shown and described herein.
